(19) United States
(12) Patent Application Publication
(10) Pub. No.: US 2026/0272357 A1
(43) Pub. Date: Sep. 17, 2026

(54) INTEGRATED PROGRAMMABLE WEARABLE DEVICE FOR HEMODYNAMIC MONITORING

(71) Applicant: University of Maryland, College Park, College Park, MD (US)

(72) Inventors: Sahil SHAH, Vienna, VA (US); Jeremy YUN, Palo Alto, CA (US)

(21) Appl. No.: 19/569,422

(22) Filed: Mar. 17, 2026

Related U.S. Application Data
(60) Provisional application No. 63/773,302, filed on Mar. 17, 2025.

(51) Int. Cl. A61B 5/28; A61B 5/00; A61B 5/11; A61B 5/256

(52) U.S. Cl. CPC A61B 5/28; A61B 5/0059; A61B 5/11; A61B 5/256; A61B 5/7285

(57) ABSTRACT

In accordance with the present disclosure, a system for monitoring hemodynamic signals of a user includes a wearable device configured to be worn on a limb of the user, the wearable device including an electrode array having one or more current generation electrodes and one or more voltage detection electrodes, one or more electrocardiogram electrodes configured to acquire biopotential signals, an optical sensing subsystem having a light-emitting device configured to emit light toward skin tissue and one or more photodiodes configured to detect reflected and/or transmitted light, an accelerometer configured to detect motion, a processor, and a memory storing instructions that, when executed by the processor, cause the wearable device to generate a photoplethysmography signal, acquire an electrocardiogram signal and an electrical bioimpedance signal, and process the signals based at least in part on detected motion to compensate for motion-related effects and generate one or more updated physiological signals.

### CROSS-REFERENCE TO RELATED APPLICATION

This application claims priority to U.S. Provisional Patent Application No. 63/773,302 filed on Mar. 17, 2025, which is incorporated herein by reference in its entirety.

### TECHNICAL FIELD

The subject matter of the present disclosure relates generally to monitoring hemodynamic signals. More particularly, the subject matter of the present disclosure relates to a multimodal wearable device capable of continuously monitoring hemodynamic signals, such as, Electrocardiogram (ECG), and Photoplethysmography (PPG), and Electrical Bioimpedance (EBI).

### BACKGROUND

Cardiovascular disease is the leading cause of death globally, with an estimated 20.5 million lives being taken each year. Heart and vascular disorders affect approximately 121.5 million adults, or 48% of individuals in the United States alone. Hemodynamically based signals such as Electrocardiogram (ECG), Photoplethysmography (PPG), and Electrical Bioimpedance (EBI) offer critical health indicators essential to modern medical diagnostics for these diseases. Non-invasive monitoring of vascular flow can offer indispensable information for assessing the health of vascular and tissue systems without the need for intervention or surgery. Collectively, these technologies enable a comprehensive evaluation of a patient's health, facilitating the early detection and management of medical conditions.

Typically, wearable device designs focus on specific hardware systems tailored to measure distinct physiological signals. A variety of commercial and academic devices are available that support the measurement of ECG and PPG. Commercially available PPG devices, for example, are engineered specifically to assess blood oxygen levels. Devices dedicated to ECG recordings primarily monitor cardiac activity. Although many ambulatory ECG monitoring systems have been commercialized to date, a major problem is still faced due to patients/athletes performing motion-related activities that introduce unwanted signal noise that makes monitoring less effective. Other key challenges identified were manual static screening, the need to learn device operations at the user's end, the effect on signal quality during real-time long-term monitoring, data processing, analysis and interpretation for the amount of data generated, sensor type and size and designs to keep it user-friendly and being biocompatible for long-term monitoring.

Moreover, advances in mobile operating systems and the emergence of artificial intelligence bring their own benefits and challenges. Specifically for those devices that measure hemodynamic parameters (including ECG), they only measure 1 parameter or 2 at most due to limitations in size and on-board computing capacity of the device. Portable devices that measure EBI have also been developed, enhancing the assessment of vascular flow and body composition. However, comprehensive patient health assessment often necessitates the simultaneous measurement of multiple physiological signals.

Furthermore, previous designs of hemodynamic measuring devices often feature large, discrete, front-end-only platforms rarely integrated onto a single device. Few possess both onboard processing and wireless transmit capabilities. Additionally, very few devices incorporate multiple bio signal sensing channels needed for comprehensive hemodynamic monitoring.

Accordingly, there remains a need for a smaller, more integrated, and power-efficient wearable platform that can simultaneously measure ECG, PPG, and EBI, with onboard processing capabilities.

### SUMMARY

In accordance with the present disclosure, a system for monitoring hemodynamic signals of a user includes a wearable device configured to be worn on a limb of the user, the wearable device including an electrode array having one or more current generation electrodes and one or more voltage detection electrodes, one or more electrocardiogram electrodes configured to acquire biopotential signals, an optical sensing subsystem having a light-emitting device configured to emit light toward skin tissue and one or more photodiodes configured to detect reflected and/or transmitted light, an accelerometer configured to detect motion, a processor, and a memory storing instructions that, when executed by the processor, cause the wearable device to generate a photoplethysmography signal, acquire an electrocardiogram signal and an electrical bioimpedance signal, and process the signals based at least in part on detected motion to compensate for motion-related effects and generate one or more updated physiological signals.

In an aspect, the system may include instructions that may temporally synchronize the electrocardiogram signal, the photoplethysmography signal, and the electrical bioimpedance signal to generate time-aligned physiological data.

In an aspect, the optical sensing subsystem may perform ambient light cancellation prior to generation of the photoplethysmography signal.

In an aspect, the accelerometer may include a multi-axis accelerometer, and processing may include compensating for motion-related effects using acceleration data measured along multiple axes.

In an aspect, the system may include instructions that may generate an impedance plethysmography signal by computing a time derivative of the electrical bioimpedance signal.

In an aspect, the system may include instructions that may calculate pulse transit time based on a temporal relationship between a feature of the electrocardiogram signal and a corresponding feature of at least one of the photoplethysmography signal or the electrical bioimpedance signal.

In an aspect, the system may include instructions that may estimate systolic blood pressure based at least in part on the calculated pulse transit time.

In an aspect, the electrode array, the light-emitting device, and the accelerometer may be co-located within a common housing of the wearable device.

In an aspect, the wearable device may be configured to be worn on a forearm with a bottom surface positioned against an inner surface of the forearm during use.

In an aspect, the wearable device may include a wireless network interface that may transmit the updated physiological signals to a mobile computing device executing an application configured to display physiological waveforms or hemodynamic metrics.

In accordance with the present disclosure, a method for monitoring hemodynamic signals using a wearable device includes illuminating skin tissue with one or more wavelengths of light, measuring reflected and/or transmitted light, generating a photoplethysmography signal, acquiring an electrocardiogram signal and an electrical bioimpedance signal, detecting motion using an accelerometer during signal acquisition, processing the signals based at least in part on detected motion to reduce motion-related artifacts and generate updated physiological signals, and transmitting the updated physiological signals to a user interface.

In an aspect, processing may include temporally synchronizing the electrocardiogram signal, the photoplethysmography signal, and the electrical bioimpedance signal to generate time-aligned physiological data.

In an aspect, the method may include performing ambient light cancellation prior to generating the photoplethysmography signal.

In an aspect, detecting motion may include measuring multi-axis acceleration and processing may include compensating for motion-related effects using the measured acceleration.

In an aspect, the method may include generating an impedance plethysmography signal by computing a time derivative of the electrical bioimpedance signal.

In an aspect, the method may include calculating pulse transit time based on a temporal relationship between a feature of the electrocardiogram signal and a corresponding feature of at least one of the photoplethysmography signal or the electrical bioimpedance signal.

In an aspect, the method may include estimating systolic blood pressure based at least in part on the calculated pulse transit time.

In an aspect, the electrode array, the light-emitting device, and the accelerometer may be co-located within a common housing of the wearable device.

In an aspect, the wearable device may be worn on a forearm with a bottom surface positioned against an inner forearm surface during use.

In an aspect, the method may include displaying updated physiological signals or hemodynamic metrics on a display of the user interface.

In an aspect, transmitting may include wirelessly transmitting the updated physiological signals to a mobile computing device executing an application configured to present physiological waveforms or trends.

Further details and aspects of exemplary embodiments of the present disclosure are described in more detail below with reference to the appended figures.

### BRIEF DESCRIPTION OF THE DRAWINGS

A better understanding of the features and advantages of the present disclosure will be obtained by reference to the following detailed description that sets forth illustrative embodiments, in which the principles of the present disclosure are utilized, and the accompanying drawings of which:

FIG. 1 is a diagram of a system for measuring and processing physiological signals, in accordance with the present disclosure;

FIG. 2 is a block diagram of a controller for the system of FIG. 1, in accordance with the present disclosure;

FIG. 3, is a schematic of a wearable device of the system of FIG. 1, in accordance with the present disclosure;

FIG. 4 is a block diagram illustrating hardware subsystems of the wearable device of the system of FIG. 1, in accordance with the present disclosure;

FIG. 4A is a top view of a wearable device including a printed circuit board and a flexible electrode array for measuring physiological signals, in accordance with the present disclosure;

FIG. 4B is a bottom view of the wearable device of FIG. 4A, illustrating placement of optical sensing components and selectable electrode contacts configured to interface with a user's skin, in accordance with the present disclosure;

FIG. 5 is a flow diagram illustrating a method for monitoring hemodynamic signals using the wearable device of FIG. 1, in accordance with the present disclosure;

FIG. 6A is a graphical illustration of a real-time ECG signal collected by the system of FIG. 1, in accordance with the present disclosure;

FIG. 6B is a graphical illustration of a real-time EBI signal collected concurrently with the ECG signal of FIG. 6A, collected by the system of FIG. 1, in accordance with the present disclosure;

FIG. 7 is a graphical illustration of a pulse wave blood flow measurement collected by the system of FIG. 1, in accordance with the present disclosure;

FIG. 8 is an exemplary graphical representation displayed on a user interface, illustrating ECG signals, bioimpedance signals, and derived blood flow metrics generated by the system of FIG. 1, in accordance with the present disclosure; and

FIG. 9 is a diagram of an exemplary overall system for measuring and processing physiological signals, in accordance with the present disclosure.

### DETAILED DESCRIPTION

The present disclosure relates generally to a multimodal wearable device capable of continuously monitoring hemodynamic signals, such as ECGs, PPG, and/or EBI.

Although the present disclosure will be described in terms of specific examples, it will be readily apparent to those skilled in this art that various modifications, rearrangements, and substitutions may be made without departing from the spirit of the present disclosure.

For the purpose of promoting an understanding of the principles of the present disclosure, reference will now be made to exemplary embodiments illustrated in the drawings, and specific language will be used to describe the same. It will nevertheless be understood that no limitation of the scope of the present disclosure is thereby intended. Any alterations and further modifications of the novel features illustrated herein, and any additional applications of the principles of the present disclosure as illustrated herein, which would occur to one skilled in the relevant art and having possession of this disclosure, are to be considered within the scope of the present disclosure.

The present disclosure relates to systems and methods for measuring and processing physiological signals, including electrocardiogram (ECG), photoplethysmography (PPG), and electrical bioimpedance (EBI), using a wearable device. ECG measurements provide information indicative of cardiac electrical activity and may be used to derive physiological markers such as heart rate, cardiac rhythm characteristics, and timing features associated with the QRS complex. In wearable implementations, ECG signals are typically acquired using reduced-lead configurations suitable for limb-worn form factors, in which electrical potentials are measured between electrodes interfacing the skin. However, ECG signals acquired in portable and wearable settings are inherently susceptible to noise and artifacts arising from motion, electrode contact variability, baseline drift, power-line interference, and physiological processes such as respiration. These effects can degrade signal fidelity, limit timing accuracy, and reduce the reliability of long-term or ambulatory ECG monitoring when ECG is used as a standalone sensing modality.

In addition to ECG, the disclosed system incorporates photoplethysmography and electrical bioimpedance sensing to enable concurrent, multi-modal physiological monitoring. PPG operates by illuminating skin tissue with one or more wavelengths of light and measuring corresponding variations in absorbed, reflected, or transmitted light caused by pulsatile blood volume changes, while EBI measures impedance variations within tissue resulting from changes in blood volume and vascular flow. Each sensing modality provides complementary physiological information but is also susceptible to motion-related and environmental artifacts when deployed independently in wearable form factors. The disclosed device provides a technical improvement over conventional wearable monitoring systems by co-locating ECG, PPG, and EBI sensing elements on a limb-worn platform and by leveraging motion sensing to reduce artifacts across all acquired signals. This coordinated, time-aligned acquisition and processing improves signal robustness, enables cross-modal validation of physiological features, and supports derivation of higher-order hemodynamic metrics—such as pulse timing relationships, blood flow-related parameters, and vascular dynamics—that are difficult to obtain accurately using any single sensing modality alone. As a result, the disclosed system enables more reliable continuous monitoring in wearable applications while maintaining a compact, low-power form factor suitable for long-term use.

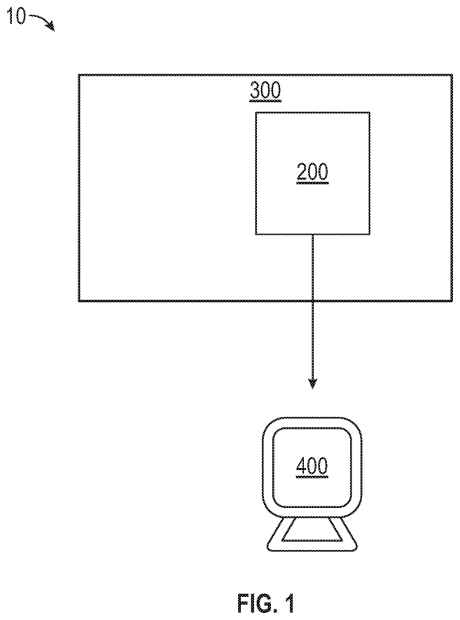

FIG. 1 illustrates a system 10 for measuring and processing physiological signals. The system 10 is configured to perform continuous, non-invasive monitoring of hemodynamic parameters of a user and to generate physiological signal data indicative of cardiovascular and vascular function. In particular, the system 10 enables concurrent acquisition and processing of multiple physiological signals, including electrocardiogram (ECG), photoplethysmography (PPG), and electrical bioimpedance (EBI), and supports wireless communication of such signals to a user-facing application.

The system 10 generally includes a wearable device 300, a controller 200, and a user interface 400. The wearable device 300 is configured to be worn on a limb of a user and includes one or more physiological sensors for acquiring physiological signal data. In some embodiments, the wearable device 300 includes an integrated electrode array, an optical sensing assembly, and one or more motion sensors, along with associated front-end acquisition circuitry. The controller 200 is disposed within the wearable device 300 and is operatively coupled to the physiological sensors. The controller 200 is configured to control signal acquisition operations, process physiological signal data acquired by the wearable device 300, and generate processed physiological signals. In some embodiments, the controller 200 performs signal conditioning, motion artifact removal, feature extraction, and/or computation of derived physiological metrics.

The user interface 400 is operatively coupled to the wearable device 300 via a wireless communication link and is configured to receive physiological signal data from the wearable device 300. The user interface 400 may be implemented on a mobile device, tablet, computer, or other external computing device, and is configured to present physiological signal information to a user. In some embodiments, the user interface 400 further enables data storage, visualization, user interaction, and communication of physiological data to remote systems or cloud-based services.

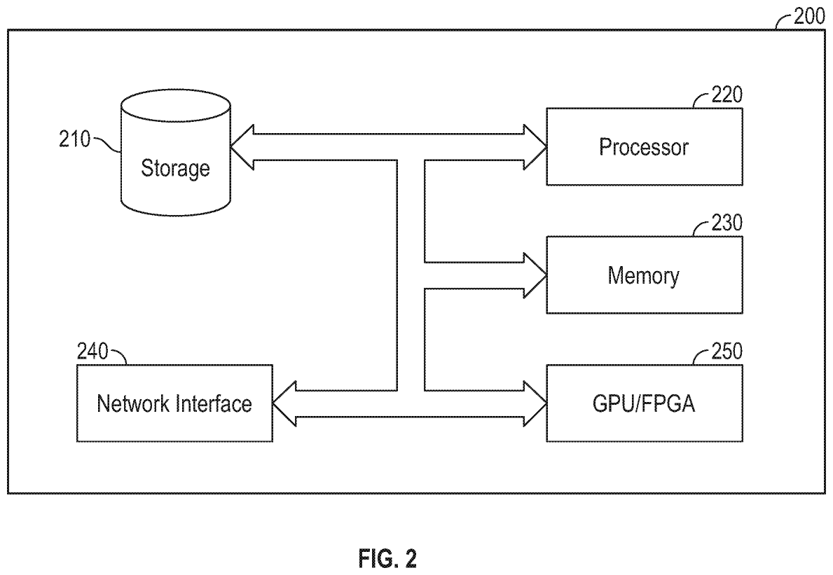

Referring now to FIG. 2, exemplary components in the controller 200 in accordance with aspects of the present disclosure include, for example, a database 210, one or more processors 220, at least one memory 230, and a network interface 240. In aspects, the controller 200 may include a graphical processing unit (GPU) 250, which may be used for processing machine learning models.

Database 210 can be located in storage. The term “storage” may refer to any device or material from which information may be capable of being accessed, reproduced, and/or held in an electromagnetic or optical form for access by a computer processor. Storage may be, for example, volatile memory such as RAM, non-volatile memory, which permanently holds digital data until purposely erased, such as flash memory, magnetic devices such as hard disk drives, and optical media such as a CD, DVD, Blu-ray disc, or the like. In various embodiments, data may be stored on the controller 200, including, for example, user preferences, historical data, and/or other data. The data can be stored in database 210 and sent via the system bus to the processor 220.

As will be described in more detail herein, the processor 220 is configured to execute instructions stored in memory 230 and to process data stored in storage 210. In some embodiments, the processor 220 controls operation of one or more sensors, performs signal conditioning and artifact removal, executes feature extraction algorithms, and generates processed physiological signals. The network interface 240 is configured to enable wired or wireless communication between the controller 200 and external devices, including a user interface device, and may support communication protocols such as Bluetooth®, Bluetooth Low Energy (BLE), Wi-Fi®, or other suitable communication standards. The illustration of FIG. 2 is exemplary, and it will be understood by persons skilled in the art that other components may exist in a controller 200. Such other components are not illustrated in FIG. 2 for clarity of illustration.

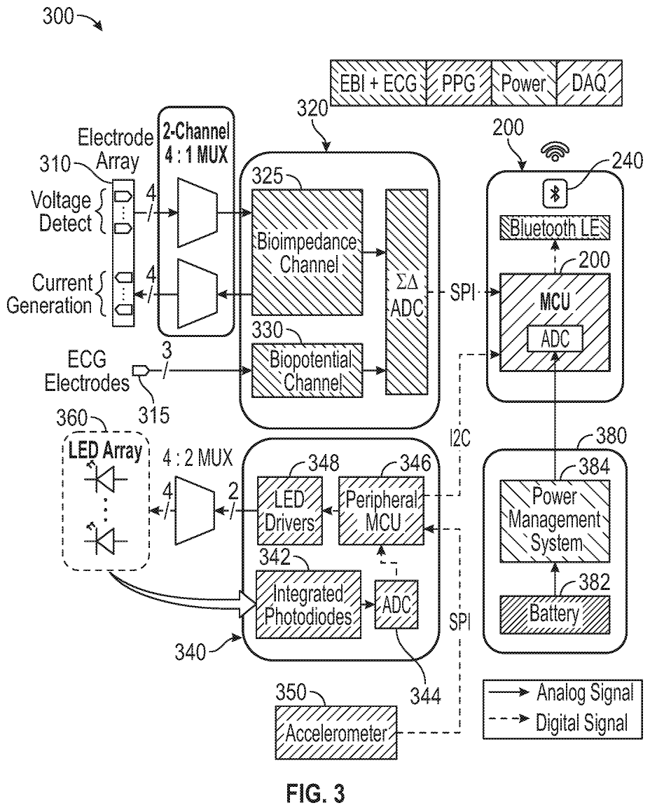

Referring now to FIG. 3, the wearable device 300 includes a plurality of integrated subsystems configured to acquire, process, and transmit physiological signals of a user. In the illustrated embodiment, the wearable device 300 includes an electrode array 310 and associated ECG electrodes 315, an electrical bioimpedance (EBI) and biopotential acquisition subsystem 320, an optical sensing subsystem 340, one or more motion sensors including an accelerometer 350, a controller 200, and a power management system 380. The wearable device 300 is configured to be worn on a limb of a user and to support continuous or intermittent monitoring of hemodynamic signals, including electrocardiogram (ECG), photoplethysmography (PPG), and electrical bioimpedance (EBI).

The electrode array 310 is configured to interface with the skin of the user and includes one or more current generation electrodes and one or more voltage detection electrodes arranged to support electrical bioimpedance measurements. In some embodiments, the electrode array 310 comprises a flexible dry electrode array fabricated on a flexible substrate or flexible printed circuit board, optionally including conductive coatings such as Ag/AgCl to improve skin contact and signal stability during long-term wear. In some aspects, the electrode array 310 is electrically coupled to the wearable device 300 via a connector, such as a ribbon connector, enabling interchangeable or custom-designed electrode configurations to accommodate different anatomical locations or use cases.

To support bioimpedance measurements across varying tissue depths and current penetration lengths, the electrode array 310 may include multiple sets of current generation and voltage detection electrodes arranged with different inter-electrode spacings, for example ranging from approximately 1 cm to 4 cm in defined increments. In some embodiments, selectable electrode pairs are coupled to downstream circuitry via one or more multiplexers to enable dynamic selection of electrode configurations during operation. For example, the pairs can be selected using a 2-channel 4:1 multiplexer (MUX).

The ECG electrodes 315 are configured to acquire biopotential signals associated with cardiac electrical activity. In some embodiments, the ECG electrodes 315 support single-lead, two-lead, or three-lead ECG configurations, with an optional reference electrode. The ECG electrodes 315 may be implemented as discrete electrodes electrically coupled to the wearable device 300, for example via a connector configured to receive commercial ECG leads or may be integrated with the electrode array 310. To obtain a measurable ECG signal, the ECG electrodes 315 may be positioned such that the positive and negative electrodes span sufficient body surface area to generate a detectable ECG signal vector, including configurations suitable for limb-worn placement.

The electrical bioimpedance and biopotential acquisition subsystem 320 is operatively coupled to the electrode array 310 and the ECG electrodes 315 and includes a bioimpedance channel 325 and a biopotential channel 330. The bioimpedance channel 325 is configured to inject an alternating current into tissue via selected electrodes of the electrode array 310 and to measure resulting voltage signals indicative of tissue impedance. In some embodiments, the magnitude and frequency of the injected current are selectable to balance tissue penetration, signal fidelity, and user safety, for example using current magnitudes in the microampere range and excitation frequencies spanning from low kilohertz to hundreds of kilohertz. The biopotential channel 330 is configured to acquire ECG signals and may include amplification, filtering, and biasing circuitry configured to condition raw ECG signals prior to digitization. In some embodiments, the ECG and EBI signals are passed through passive or active low-pass filters prior to analog-to-digital conversion to reduce high-frequency noise and aliasing.

In some embodiments, the bioimpedance channel 325 and the biopotential channel 330 each include analog-to-digital conversion circuitry configured to digitize acquired signals with sufficient resolution for downstream processing. For example, the bioimpedance channel 325 and the biopotential channel 330 may include separate sigma-delta analog-to-digital converters having different resolutions selected based on the respective signal characteristics. In some embodiments, internal DC lead biasing circuitry is used to drive the connected tissue to an appropriate common-mode voltage level when using a two-electrode or single-lead configuration, thereby maintaining electrode voltages within an input range of the acquisition circuitry without requiring a dedicated right-leg drive electrode. Digitized EBI and ECG signals are communicated to the controller 200 for further processing, including signal conditioning, artifact removal, feature extraction, and computation of derived physiological metrics.

In some embodiments, an ECG front-end input associated with the biopotential channel 330 is accessed through a connector configured to receive commercial ECG leads, such as a 3.5 mm connector jack, and is operable with a set of two or three ECG electrodes 315, with a third reference electrode being optional. To obtain a clear ECG signal, the positive and negative ECG electrodes 315 are positioned such that they span sufficient body surface area to generate a measurable ECG signal vector, thereby providing a sufficient potential difference to be detected by the biopotential channel 330. Such configurations enable reliable ECG acquisition in limb-worn wearable implementations.

In some embodiments, acquired ECG and EBI signals are passed through passive low-pass filters prior to analog-to-digital conversion to reduce high-frequency noise and aliasing. The filtered ECG and EBI signals may then be acquired by an integrated biopotential and bioimpedance analog front end within the electrical bioimpedance and biopotential acquisition subsystem 320. In some aspects, the bioimpedance channel 325 and the biopotential channel 330 include respective analog-to-digital converters having different resolutions selected based on signal characteristics, for example sigma-delta converters configured to provide higher resolution for impedance measurements and lower resolution for biopotential measurements. In some embodiments, the acquisition circuitry operates from a regulated low-voltage supply, such as approximately 1.8 volts, provided by the power management system 380. In exemplary aspects, the acquired ECG and EBI signals are passed through passive low pass filters before being acquired by an analog front end. Both the ECG and EBI channels have their own respective 18-bit and 20-bit ΣΔ analog-to-digital converters (ADC's). The analog front end is supplied with a constant 1.8V VDD level.

For both ECG and EBI signal acquisition, some embodiments employ internal DC lead biasing to drive connected tissue to an appropriate common-mode voltage level when using a single-lead or two-electrode configuration. In such embodiments, common-mode voltage of the positive and negative electrodes is body biased to an average DC level (V_{MID}), which can be calculated as

$${V}_{MID} = \frac{{V}_{D D}}{2.15} = 837 mV .$$

In some embodiments, this biasing results in an electrode input voltage range that is band-limited to approximately V_{MID}±550 mV, thereby maintaining electrode voltages within saturation limits of the acquisition circuitry. For EBI signal acquisition, the magnitude and frequency of the injected alternating current may be selectable, for example with current magnitudes ranging from approximately 8 μA to 96 μA and excitation frequencies ranging from approximately 125 Hz to 128 kHz, to balance tissue penetration, signal fidelity, and user safety.

The optical sensing subsystem 340 is configured to perform photoplethysmography (PPG) measurements and to monitor one or more physiological parameters including pulse heart rate (HR) and pulse blood oxygen saturation (SpO₂). The optical sensing subsystem 340 includes a light-emitting device 360, integrated photodiodes 342, LED drivers 348, an analog-to-digital converter 344, and a peripheral microcontroller 346. In some embodiments, the light-emitting device 360 includes a plurality of light emitters configured to emit light at different wavelengths, such as one or more green LEDs for heart rate monitoring and one or more red and infrared LEDs for SpO₂ estimation. In some aspects, the Light-emitting device 360 includes multiple LEDs emitting at nominal wavelengths of approximately 536 nm, 655 nm, and 940 nm. Selection between different LED wavelength combinations may be performed using one or more multiplexers, such as a peripheral multi-channel multiplexer, to support different PPG measurement modes.

Light emitted by the light-emitting device 360 is directed toward tissue of the user, and light reflected from or transmitted through the tissue is detected by the integrated photodiodes 342. In aspect, the light-emitting device 360 may be an LED array. Electrical signals generated by the photodiodes 342 are digitized by the ADC 344 and processed by the peripheral microcontroller 346. In some embodiments, the peripheral microcontroller 346 includes an internal processor configured to execute preprocessing operations on the acquired PPG signals, including ambient light cancellation, pulse-width-modulated (PWM) LED control, demodulation, and noise reduction. In some aspects, the optical sensing subsystem 340 includes internal LED drivers 348 with configurable drive current and timing parameters. The optical sensing subsystem 340 may further be operatively coupled to the accelerometer 350, for example via a serial communication interface, enabling motion data to be used by the peripheral microcontroller 346 or the controller 200 to reduce motion-induced artifacts in PPG signals prior to further processing or transmission.

The wearable device 300 further includes an accelerometer 350 configured to detect motion of the user during signal acquisition. Motion data generated by the accelerometer 350 is communicated to the controller 200 and may be used to identify and mitigate motion-induced artifacts in ECG, EBI, and PPG signals. In some embodiments, motion data is used to adapt signal processing parameters or to selectively discard corrupted data segments.

The wearable device 300 includes a power management system 380 configured to supply power to the various subsystems of the wearable device. The power management system 380 includes a battery 382 and power management circuitry 384 configured to regulate voltage levels supplied to analog, digital, and optical components. In some embodiments, the power management system 380 supports battery charging, power monitoring, and power-saving operating modes to enable extended wearable operation. some embodiments, the majority of onboard device components operate at an energy-efficient supply voltage of approximately 1.8 volts, while certain components, such as the light-emitting device 360 of the optical sensing subsystem 340, are supplied by a higher voltage rail generated using a step-up boost converter.

In some embodiments, the power management circuitry 384 includes a power management integrated circuit (PMIC) having an integrated DC/DC regulator configured to generate a regulated low-voltage supply for system operation and to support battery charging functionality. The battery 382 may include a single-use coin cell battery, such as a 3-volt battery, or a rechargeable battery, such as a lithium-ion coin cell battery. Where a rechargeable battery is used, the power management system 380 may include charging circuitry configured to limit charging current and to support recharging via an external interface, such as a wired charging connection.

The power management system 380 may further include battery monitoring circuitry configured to measure battery voltage, temperature, or other parameters. Battery status information may be communicated to the controller 200, which may process the sampled battery data to provide low-battery indications, adapt operating modes, or manage power consumption. In some embodiments, the wearable device 300 is configured to operate in low-power duty-cycled modes in which physiological measurements are performed intermittently, thereby enabling extended operational lifetimes. For example, while continuous operation of all subsystems may result in relatively high instantaneous current consumption, intermittent measurement operation may enable the wearable device 300 to operate for extended periods ranging from weeks to months between battery replacement or recharging, depending on the selected battery type and usage profile.

The wearable device 300 includes a controller 200 implemented as a microcontroller unit (MCU) operatively coupled to the electrical bioimpedance and biopotential acquisition subsystem 320, the optical sensing subsystem 340, the accelerometer 350, and the power management system 380. The controller 200 is configured to control sensor operation, coordinate signal acquisition, and execute signal processing algorithms. In some embodiments, the controller 200 performs motion artifact removal, feature extraction, and computation of derived physiological metrics based on ECG, EBI, and PPG signals.

The controller 200 further includes or is operatively coupled to a network interface 240 configured to wirelessly transmit acquired or processed physiological data to an external user interface device. The network interface 240 may support Bluetooth®, Bluetooth Low Energy (BLE), or other suitable wireless communication protocols. In some embodiments, the controller 200 is implemented as a microcontroller unit (MCU) that includes a multiprotocol wireless system-on-chip (SoC) having an integrated processor, memory, and radio transceiver. In some aspects, the MCU comprises a 32-bit processor core with floating-point support, internal analog-to-digital conversion circuitry, non-volatile program memory, and volatile memory sufficient to support real-time signal processing and wireless communication.

In some embodiments, the MCU is integrated within a wireless communication module that includes an onboard antenna to facilitate wireless transmission. The controller 200 may receive digitized ECG and EBI data from the electrical bioimpedance and biopotential acquisition subsystem 320 via a serial communication interface, such as a serial peripheral interface (SPI), and may receive PPG data from the optical sensing subsystem 340 via a different serial interface, such as an inter-integrated circuit (I²C) interface. In some embodiments, the controller 200 is configured with a pinout and software environment compatible with standard embedded development platforms, enabling simplified firmware development, programming, and system integration.

In operation, and with reference to FIG. 3, the wearable device 300 is configured to be worn on a limb of a user and to concurrently acquire electrocardiogram (ECG), photoplethysmography (PPG), and electrical bioimpedance (EBI) signals. The wearable device 300 operates by coordinating the electrode array 310, ECG electrodes 315, the electrical bioimpedance and biopotential acquisition subsystem 320, the optical sensing subsystem 340, the accelerometer 350, and the controller 200 to generate time-aligned physiological signals suitable for real-time monitoring and derivation of hemodynamic metrics.

The ECG signals are acquired using the ECG electrodes 315 operatively coupled to the biopotential channel 330 of the electrical bioimpedance and biopotential acquisition subsystem 320. In some embodiments, the ECG electrodes 315 are arranged in a reduced-lead configuration suitable for limb-worn placement, such as a single-lead or two-electrode configuration. The biopotential channel 330 measures a potential difference between selected ECG electrodes 315 corresponding to cardiac electrical activity and generates a digitized ECG waveform. The controller 200 processes the acquired ECG signal to identify cardiac features, including detection of R-peaks within the QRS complex. In some embodiments, the ECG signal is used to compute R-to-R intervals and heart rate, and to provide timing reference points for synchronization with concurrently acquired PPG and EBI signals. Motion data acquired from the accelerometer 350 may be used by the controller 200 to identify motion-corrupted ECG segments and to remove or mitigate motion-related artifacts.

Concurrently with ECG acquisition, the optical sensing subsystem 340 operates to acquire photoplethysmography (PPG) signals. During operation, the light-emitting device 360 emits light toward tissue of the user at one or more selected wavelengths. In some embodiments, green wavelengths are used to acquire pulse signals associated with heart rate, while red and infrared wavelengths are used to acquire signals associated with blood oxygen saturation. Light reflected from or transmitted through the tissue is detected by the integrated photodiodes 342 and converted into electrical signals. The detected optical signals are digitized and processed by the peripheral microcontroller 346 and/or the controller 200 to generate a PPG waveform indicative of pulsatile blood volume changes. In some embodiments, the optical sensing subsystem 340 performs ambient light cancellation, LED modulation and demodulation, and noise reduction prior to transmission of PPG data to the controller 200. Motion data from the accelerometer 350 may be used to compensate for motion-induced artifacts in the PPG signal.

During operation, the bioimpedance channel 325 of the electrical bioimpedance and biopotential acquisition subsystem 320 injects an alternating current into tissue of the user via selected current generation electrodes of the electrode array 310 positioned over or adjacent to a target vascular region. As pulsatile blood flow causes time-varying changes in tissue impedance, corresponding voltage differences are measured using one or more voltage detection electrodes of the electrode array 310 and digitized to produce an electrical bioimpedance (EBI) signal. To ensure sufficient tissue penetration while maintaining user comfort and safety, the injected current is selected to have a relatively high frequency and low magnitude. In some embodiments, the injection current frequency lies within a range of approximately 10 kHz to 100 kHz, and the injected current magnitude is maintained below a perception threshold defined as Iₘₐₓ=10⁻⁷*freq such that the injected current remains imperceptible to the user. In some embodiments, the bioimpedance channel 325 further employs phase-sensitive detection to extract both resistive and reactive components of tissue impedance. The acquired EBI signal is subsequently processed to generate an impedance plethysmography (IPG) signal representing changes in tissue impedance associated with pulsatile blood flow. The IPG signal is obtained as the time derivative of the measured EBI signal according to:

$$IPG = \frac{dEBI}{dt} \qquad ( Eqn . 1 )$$

Using the IPG signal, pulsatile blood volume ΔV_{bd} may be estimated according to:

$$Δ {V}_{b d} = {ρ ( \frac{L}{{R}_{d c}} )}^{2} Δ {{t}_{pulse} ( \frac{dEBI}{dt} )}_{ma x} \qquad ( Eqn . 2 )$$

where ρ is blood resistivity, L is the distance between voltage electrodes, R_{dc} is the DC resistance of tissue, Δtpulse is the time difference between the minima preceding and following each EBI pulse, and

$${( \frac{dEBI}{dt} )}_{m ax}$$

is the rising edge amplitude of the IPG signal.

Finally, the blood flow rate (Qbd) is calculated using Eqn. 3, where HR corresponds to heart rate.

$${Q}_{b d} = Δ {V}_{b d} \cdot HR \qquad ( Eqn . 3 )$$

As such, to calculate blood flow rate, EBI must be measured concurrently with either the systolic peaks from PPG or R-to-R detection from ECG.

In operation, ECG, PPG, and EBI signals are acquired concurrently and are time-aligned by the controller 200. The concurrent acquisition enables correlation of cardiac electrical activity, peripheral pulse waveforms, and impedance-based blood volume changes. In some embodiments, the controller 200 calculates pulse transit time (PTT) based on a time difference between an R-peak detected in the ECG signal and a corresponding systolic feature detected in the PPG signal, which may be used to estimate systolic blood pressure or other hemodynamic parameters.

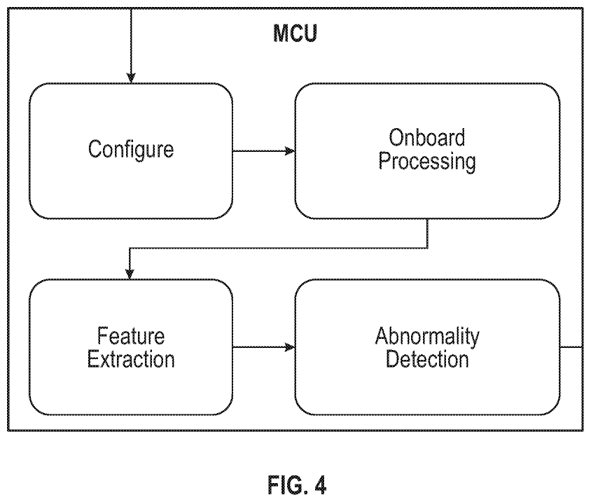

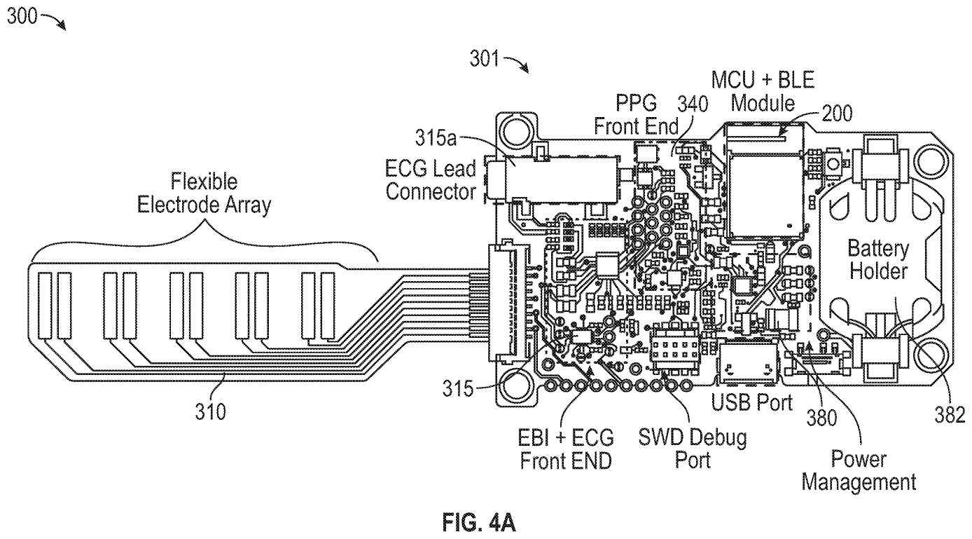

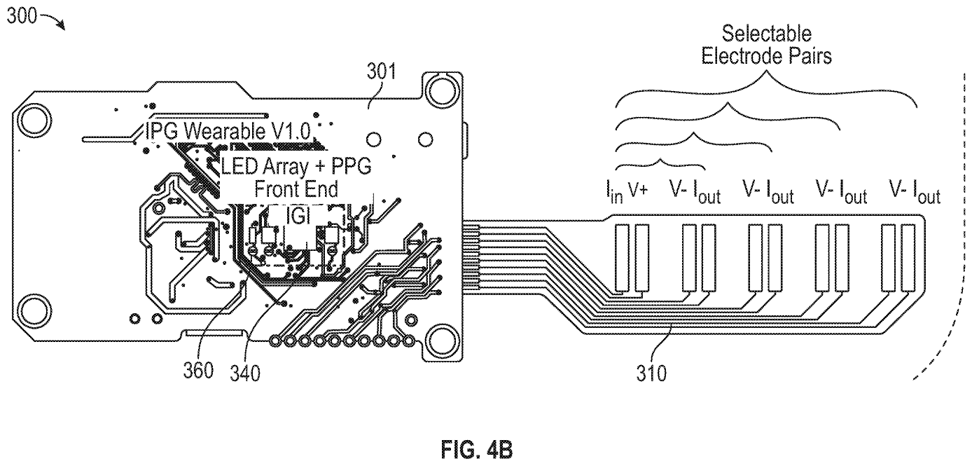

FIG. 4 is a block diagram illustrating exemplary hardware subsystems of the wearable device of the system of FIG. 1, in accordance with the present disclosure.

FIGS. 4A and 4B illustrate a physical embodiment of the wearable device 300 in accordance with aspects of the present disclosure. FIG. 4A shows a top view of the wearable device 300, and FIG. 4B shows a bottom view of the wearable device 300. In the illustrated embodiment, the wearable device 300 comprises a rigid printed circuit board (PCB) portion 301 housing electronic components and a flexible electrode array 310 extending from the rigid PCB to enable conformal contact with a user's skin when the device is worn on a limb, such as a forearm.

Referring to FIG. 4A, the wearable device 300 includes a rigid PCB portion 301 coupled to the flexible electrode array 310. The flexible electrode array 310 extends outward from the rigid PCB and includes a plurality of electrode pads arranged to form selectable electrode pairs for electrical bioimpedance and biopotential measurements. The flexible electrode array 310 may be fabricated on a flexible substrate to allow the electrode pads to conform to the curvature of the user's limb during wear.

The rigid PCB portion 301 of the wearable device 300 supports electronic subsystems including the controller 200, the optical sensing subsystem 340, and the power management system 380. In the illustrated embodiment, an ECG lead connector 315a is disposed on the rigid PCB and is configured to electrically couple external ECG electrodes 315 to the wearable device 300. The power management system 380 is arranged adjacent to a battery holder configured to receive a battery 382 for powering the wearable device 300. In some embodiments, the rigid PCB portion 301 further includes one or more external connectors or ports to facilitate device programming, debugging, charging, or data transfer.

Referring to FIG. 4B, the bottom view of the wearable device 300 illustrates components positioned on an opposing side of the rigid PCB portion 301 from those shown in FIG. 4A. In some embodiments, the bottom face of the wearable device 300 is positioned against an inner surface of a forearm of the user during wear, such that the optical sensing subsystem 340 and associated components are oriented toward the skin. In the illustrated embodiment, the optical sensing subsystem 340 is disposed on the bottom surface of the rigid PCB portion 301 such that optical components are positioned to face the user's skin during use. The light-emitting device 360 of the optical sensing subsystem 340 is arranged to emit light toward the skin, while associated optical sensing components are positioned to receive light reflected from or transmitted through underlying tissue.

The bottom view further illustrates the flexible electrode array 310 extending from the rigid PCB portion 301 and including multiple electrode pads arranged as selectable electrode pairs. The electrode pads may be configured to serve as current generation electrodes and voltage detection electrodes for electrical bioimpedance measurements, as well as to support biopotential measurements. The spatial separation and layout of the electrode pads on the flexible electrode array 310 enable selection of different electrode pair spacings to accommodate different tissue depths, anatomical locations, or measurement modalities.

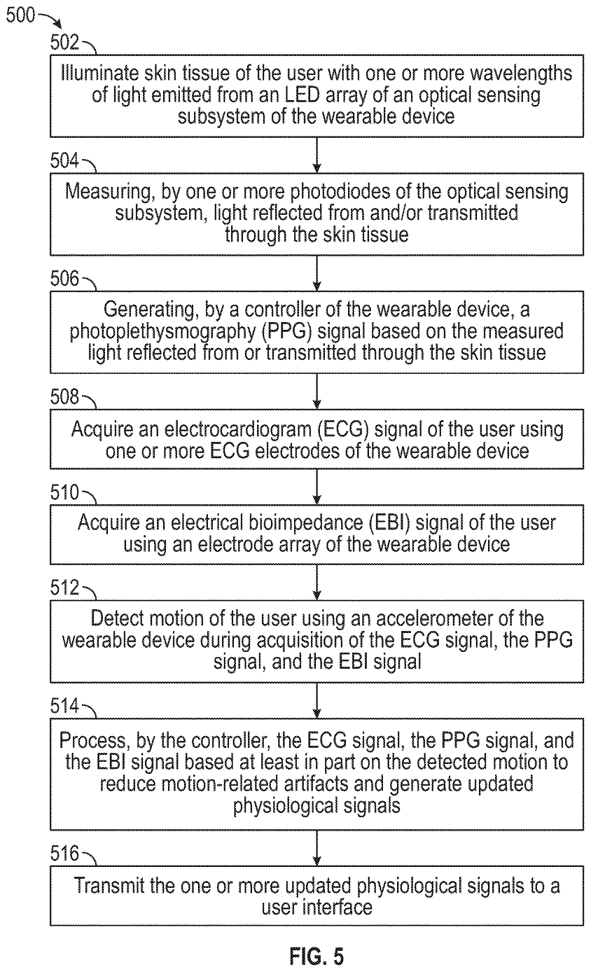

FIG. 5 illustrates an example method 500 for monitoring physiological and hemodynamic signals using the wearable device 300 described herein (e.g., FIGS. 1-4). The method 500 may be executed by coordinated operation of the electrode array 310, ECG electrodes 315, electrical bioimpedance and biopotential acquisition subsystem 320 (including the bioimpedance channel 325 and biopotential channel 330), the optical sensing subsystem 340 (including the integrated photodiodes 342, ADC 344, peripheral microcontroller 346, LED drivers 348, and light-emitting device 360), the accelerometer 350, the controller 200, and the network interface 240. In example embodiments, method 500 enables concurrent or substantially concurrent acquisition of ECG, PPG, and EBI signals and generation of motion-compensated (updated) physiological signals suitable for real-time monitoring and for computation of derived parameters, such as heart rate, blood oxygen saturation, impedance plethysmography (IPG), pulse transit time (PTT), or other hemodynamic metrics. Although the steps of method 500 are illustrated in a particular order, it will be understood that one or more steps may be performed in a different order, performed concurrently, combined, repeated, or omitted in alternative embodiments without departing from the scope of the disclosure.

At step 502, skin tissue of the user is illuminated with one or more wavelengths of light emitted from the light-emitting device 360 of the optical sensing subsystem 340 of the wearable device 300. The light-emitting device 360 is driven by LED drivers 348, which may control LED current, pulse width, duty cycle, modulation frequency, and timing to improve optical signal quality and reduce power consumption. In some embodiments, the light-emitting device 360 includes multiple LEDs having different emission spectra, enabling selection among wavelengths based on the intended physiological measurement (e.g., pulse waveform monitoring and/or oxygenation monitoring).

In some embodiments, illumination is performed according to a timing schedule in which different wavelengths are emitted sequentially, interleaved, or multiplexed. For example, the peripheral microcontroller 346 and/or controller 200 may control the light-emitting device 360 to emit at a first wavelength during a first time slot and at a second wavelength during a second time slot, thereby enabling wavelength-specific PPG processing. In some embodiments, illumination parameters are adapted based on a detected ambient light condition, a measured photodiode signal level, a skin-contact condition, a motion condition from the accelerometer 350, and/or a battery status associated with the power management system 380.

At step 504, light reflected from and/or transmitted through the illuminated skin tissue is measured by the integrated photodiodes 342 of the optical sensing subsystem 340. The photodiodes 342 generate an electrical signal representative of received optical energy that varies as tissue optical properties change, including changes due to pulsatile blood volume. In some embodiments, the optical sensing subsystem 340 uses two or more photodiodes 342 to improve robustness to motion, improve signal-to-noise ratio, support spatial averaging, or enable selection among photodiodes based on signal quality. In some embodiments, the ADC 344 digitizes one or more photodiode signals at a sampling rate selected to capture pulsatile dynamics, and the digitized samples are timestamped or otherwise temporally referenced to illumination timing events (e.g., LED on/off intervals) to facilitate synchronous detection. In some embodiments, the optical sensing subsystem 340 adjusts gain, integration time, or sampling parameters to avoid saturation, maintain dynamic range, or accommodate different skin tones, tissue compositions, or placement sites.

At step 506, a PPG signal is generated based on the measured optical signals. In some embodiments, the peripheral microcontroller 346 performs preprocessing operations on digitized photodiode signals prior to transmitting data to the controller 200. Preprocessing may include, in some embodiments, ambient light cancellation, demodulation of LED-modulated signals, baseline correction, filtering, noise suppression, normalization, and/or generation of signal quality indices. In some embodiments, the controller 200 generates a PPG waveform indicative of pulsatile blood volume changes and may further extract features from the PPG waveform, including pulse peaks, pulse intervals, waveform rise time, waveform morphology features, and/or perfusion indicators. In some embodiments, the optical sensing subsystem 340 operates in a first mode to generate a heart-rate-oriented PPG waveform and in a second mode to generate multi-wavelength optical measurements usable for estimation of blood oxygen saturation (SpO₂). In some embodiments, the controller 200 selects among available wavelengths or photodiode channels based on signal quality, detected motion, or a user-selected operating mode.

At step 508, an ECG signal is acquired using one or more ECG electrodes 315 operatively coupled to the biopotential channel 330 of the electrical bioimpedance and biopotential acquisition subsystem 320. In some embodiments, the ECG electrodes 315 are arranged as a reduced-lead configuration suitable for limb-worn placement, such as a two-electrode (single-lead) configuration or a three-electrode configuration including an optional reference electrode. In some embodiments, the biopotential channel 330 conditions the ECG signal using one or more analog stages including amplification and filtering prior to digitization. The digitized ECG waveform may be communicated to the controller 200 for subsequent processing. In some embodiments, the controller 200 identifies cardiac features such as R-peaks, computes R-to-R intervals, generates heart rate estimates, and/or generates beat-to-beat timing markers that are used to align ECG measurements with PPG and EBI measurements. In some embodiments, the ECG signal is further evaluated to identify motion corruption, electrode contact degradation, or other signal-quality conditions.

At step 510, an EBI signal is acquired using the electrode array 310 in combination with the bioimpedance channel 325 of the electrical bioimpedance and biopotential acquisition subsystem 320. In some embodiments, the electrode array 310 includes multiple electrode pairs enabling selection among different current injections and voltage sensing geometries to accommodate different limb sizes, different placement positions, or different tissue penetration depths.

In some embodiments, the bioimpedance channel 325 injects an alternating current into tissue via one or more current generation electrodes and measures a resulting voltage using one or more voltage detection electrodes to determine tissue impedance. In some embodiments, the injection frequency is selected within a range suitable for tissue penetration and user comfort, and the injection amplitude is selected to comply with applicable safety thresholds. In some embodiments, the bioimpedance channel 325 uses phase-sensitive detection to extract resistive and reactive impedance components and outputs a digitized EBI waveform to the controller 200. In some embodiments, the controller 200 generates an impedance plethysmography (IPG) signal based on the EBI signal, including by computing a time derivative of the EBI waveform to emphasize pulsatile impedance changes associated with blood flow. The controller 200 may additionally extract EBI/IPG features such as pulse amplitude, pulse timing, rise slope, and beat-to-beat variability.

At step 512, motion of the user is detected using the accelerometer 350 of the wearable device 300 during acquisition of the ECG, PPG, and EBI signals. In some embodiments, the accelerometer 350 provides multi-axis acceleration samples and/or derived motion features, such as acceleration magnitude, activity classification, or motion intensity. In some embodiments, accelerometer data are synchronized with one or more of the ECG, PPG, and EBI sample streams so that motion events can be associated with specific physiological signal segments. In some embodiments, the controller 200 uses accelerometer data to detect motion events that are likely to induce artifacts (e.g., sudden impacts, periodic arm movement, or device repositioning), and to label or characterize affected portions of the physiological waveforms.

At step 514, the controller 200 processes the ECG, PPG, and EBI signals based at least in part on the detected motion to reduce motion-related artifacts and generate updated physiological signals. In some embodiments, the controller 200 performs motion-aware filtering, adaptive noise cancellation, artifact detection and removal, segment rejection, interpolation, weighting, or other signal conditioning operations using the accelerometer 350 as a reference input. The output may include updated (motion-compensated) ECG, PPG, and EBI/IPG waveforms. In some embodiments, the controller 200 performs multimodal synchronization and fusion, including aligning cardiac timing markers from the ECG (e.g., R-peak timing) with corresponding pulse features in the PPG and/or IPG waveforms. In some embodiments, the controller 200 derives hemodynamic parameters from time-aligned signals, such as pulse transit time (PTT) computed from a time difference between an ECG feature and a corresponding PPG or IPG feature. In some embodiments, the controller 200 estimates blood pressure (e.g., systolic blood pressure) based at least in part on the PTT and/or additional features extracted from the updated physiological signals. In some embodiments, the controller 200 computes signal quality indices for one or more channels and uses those indices to control operation of the wearable device 300, including adapting LED drive, adjusting sampling parameters, selecting electrode configurations, or determining whether to transmit raw versus processed data.

At step 516, one or more updated physiological signals and/or derived parameters are transmitted via the network interface 240 to a user interface 400. In some embodiments, the network interface 240 comprises a Bluetooth® or Bluetooth Low Energy (BLE) interface configured to transmit data to a mobile device executing an application that presents waveforms, metrics, and/or alerts. In some embodiments, the transmitted data include time-series waveforms (e.g., updated ECG, updated PPG, updated EBI/IPG), extracted features (e.g., R-peak timestamps, pulse peak timestamps), derived hemodynamic parameters (e.g., heart rate, SpO₂, PTT, blood pressure estimates, blood flow estimates), and/or quality indicators. In some embodiments, the user interface 400 displays graphical outputs of the waveforms and/or computed parameters, stores session history, or forwards data to external systems for remote review. In some embodiments, the controller 200 manages buffering, packetization, encryption, or retry logic for wireless communication via the network interface 240, and may transmit data continuously, periodically, or in response to detected events.

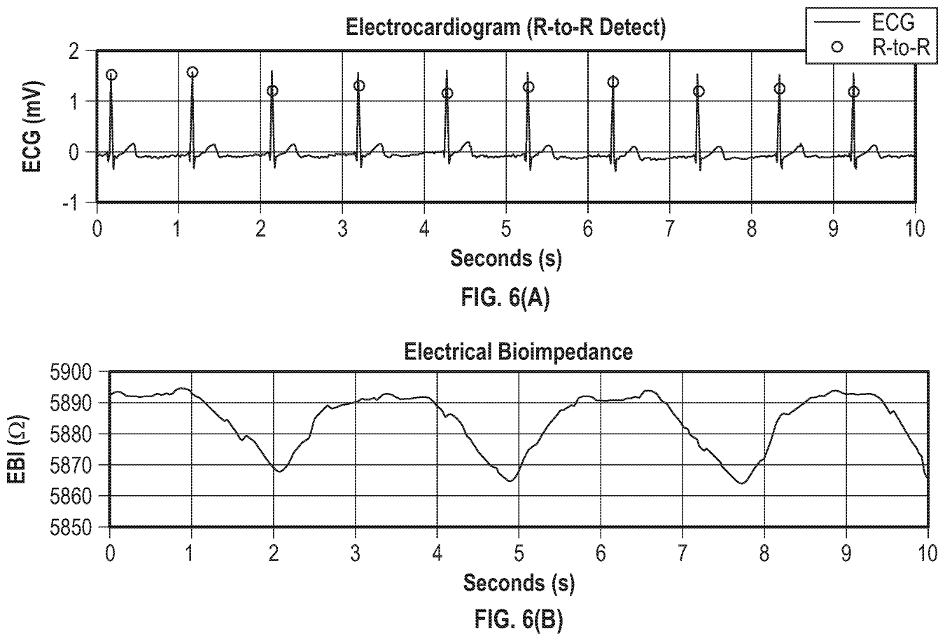

FIGS. 6A and 6B illustrate example results corresponding to real-time, concurrent acquisition of electrocardiogram (ECG) and electrical bioimpedance (EBI) signals generated by the wearable device described herein. In an aspect of the present disclosure, the bio signal acquisition channels were validated using commercially available evaluation platforms corresponding to the bio signal front-end components employed by the wearable device. For ECG and EBI acquisition, an analog front end evaluation platform was used to evaluate performance of the analog front end biopotential and bioimpedance front end under representative operating conditions. These validation tests demonstrate proof-of-concept functionality of the ECG and EBI acquisition channels and do not limit the disclosed wearable device architecture, which integrates the described subsystems into a compact, limb-worn form factor.

FIG. 6A illustrates an example ECG waveform acquired in real time, including active R-to-R interval detection. In this demonstration, the ECG signal was acquired using a reduced-lead configuration representative of limb-worn operation, with electrodes positioned to capture cardiac electrical activity suitable for heart rate and timing analysis. The resulting waveform demonstrates clear detection of R-peaks and corresponding R-to-R intervals, as identified by the biopotential acquisition channel and subsequent signal processing. The detected cardiac timing markers may be used directly for heart rate estimation and may further serve as synchronization references for concurrently acquired PPG and EBI signals, as described elsewhere in the present disclosure.

FIG. 6B illustrates an example EBI waveform acquired concurrently with the ECG signal. In this demonstration, an alternating current having a magnitude of approximately 8 μA and an excitation frequency of approximately 16 kHz was injected into tissue using selected electrode pairs, and resulting voltage changes were measured to generate an EBI signal indicative of impedance variations associated with physiological activity. The illustrated waveform reflects periodic impedance changes corresponding to pulsatile blood volume variations within peripheral tissue. In embodiments of the disclosed wearable device, such EBI signals may be further processed to generate impedance plethysmography (IPG) waveforms, which can be synchronized with ECG and PPG signals to support estimation of hemodynamic parameters such as blood flow metrics, pulse timing, and pulse transit time (PTT).

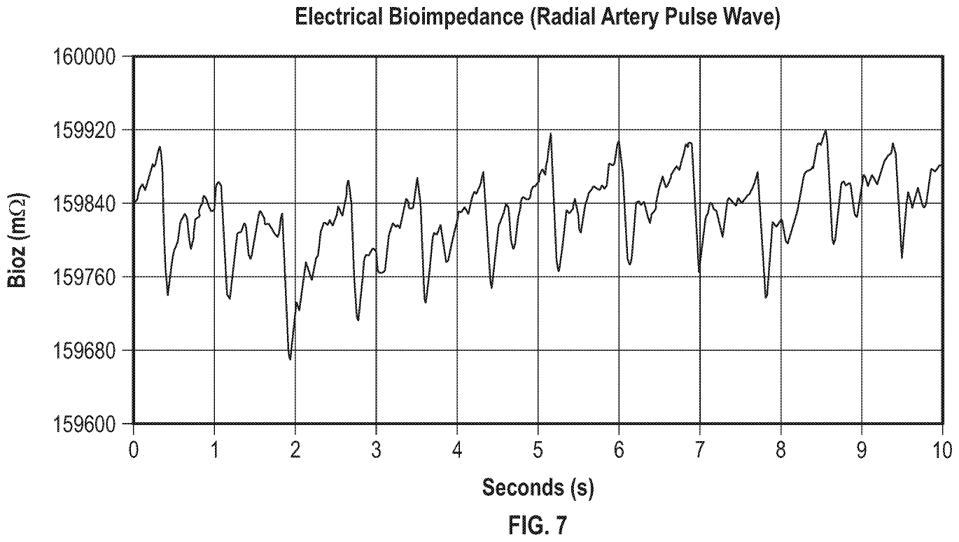

FIG. 7 illustrates an example graphical representation of an electrical bioimpedance (EBI) signal acquired using the wearable device 300 positioned on a limb of a user, such as an inner surface of a forearm, in accordance with the present disclosure. In the illustrated embodiment, the EBI signal is acquired using the electrode array 310 and the bioimpedance channel 325 of the electrical bioimpedance and biopotential acquisition subsystem 320, with the electrodes positioned to span peripheral vasculature including the radial artery.

The illustrated waveform represents temporal variations in measured bioimpedance corresponding to pulsatile blood volume changes associated with the radial artery pulse wave. The graph depicts fluctuations in electrical bioimpedance (BioZ), expressed in micro-ohms (m (2), as a function of time in seconds, and reflects dynamic impedance changes occurring during successive cardiac cycles. Such impedance variations arise from periodic changes in arterial cross-sectional area and blood volume during systole and diastole, which modulate the effective electrical impedance of the underlying tissue.

In some embodiments, the acquired EBI waveform may be further processed by the controller 200 to generate an impedance plethysmography (IPG) signal, for example by computing a time derivative of the measured EBI signal, and may be temporally aligned with concurrently acquired ECG and/or PPG signals. The resulting bioimpedance-derived pulse waveform may be used to extract hemodynamic features, including pulse timing, relative pulse amplitude, pulse morphology, or vascular compliance indicators. Accordingly, FIG. 7 demonstrates the ability of the disclosed wearable device to noninvasively capture peripheral arterial pulse dynamics using electrical bioimpedance measurements, supporting continuous or periodic monitoring of cardiovascular and hemodynamic parameters.

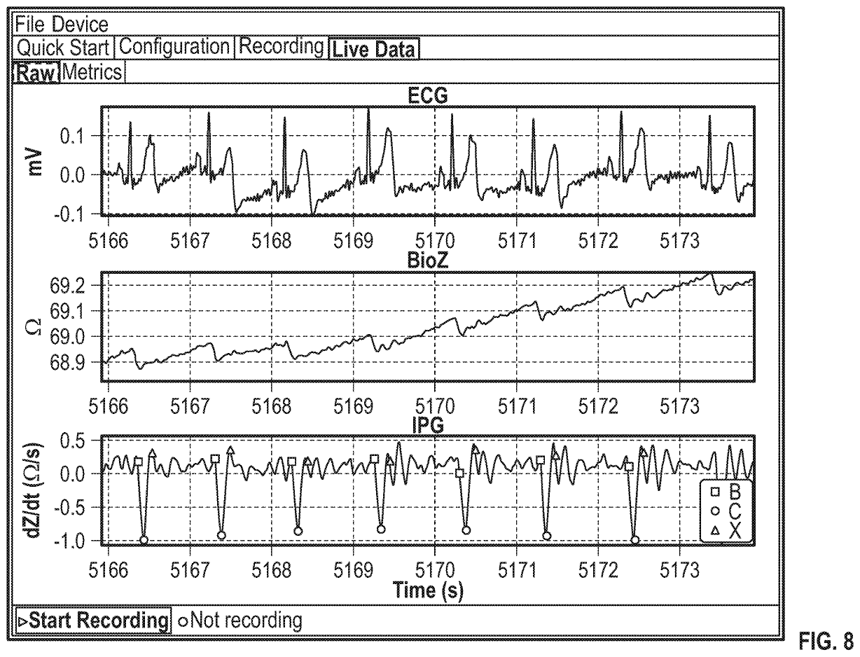

FIG. 8 illustrates example graphical representations of physiological signals and derived metrics presented on the user interface 400 communicatively coupled to the wearable device 300, in accordance with aspects of the present disclosure. In some embodiments, the user interface 400 is implemented on a smartphone, tablet, dedicated display, computer, or other external computing device configured to receive physiological data from the wearable device 300 via the network interface 240 and to render one or more visualizations corresponding to acquired and processed signals. In the illustrated embodiment, FIG. 8 shows an exemplary interface in which an electrocardiogram (ECG) waveform, an electrical bioimpedance (BioZ) waveform, and one or more derived flow-related metrics are displayed together on a common screen.

The ECG waveform shown in FIG. 8 may be generated from ECG signals acquired using the ECG electrodes 315 and the biopotential channel 330 of the electrical bioimpedance and biopotential acquisition subsystem 320. The ECG waveform may be sampled at a predetermined sampling rate, such as approximately 128 samples per second, and displayed as a time-varying signal representing cardiac electrical activity. In some embodiments, the ECG waveform is processed by the controller 200 to identify cardiac features, including R-peaks and R-to-R intervals, which may be used to compute heart rate or to provide timing reference points for synchronization with other physiological signals. The displayed ECG waveform may represent raw data, filtered data, or motion-compensated data following artifact reduction based on motion information obtained from the accelerometer 350.

The BioZ waveform shown in FIG. 8 may correspond to bioimpedance measurements acquired using the electrode array 310 and the bioimpedance channel 325 of the electrical bioimpedance and biopotential acquisition subsystem 320. The BioZ waveform represents temporal variations in measured tissue impedance and may be sampled at a lower rate relative to the ECG waveform, such as approximately 64 samples per second. In some embodiments, the BioZ signal reflects pulsatile impedance changes associated with peripheral arterial blood flow, such as blood volume changes occurring within the radial artery when the wearable device 300 is positioned on the wrist or forearm of the user. Under conditions of minimal motion, the BioZ waveform may exhibit periodic features corresponding to arterial pulse cycles, enabling extraction of hemodynamic information. The displayed BioZ waveform may represent raw impedance measurements or processed signals following filtering, normalization, or motion artifact mitigation performed by the controller 200.

FIG. 8 further illustrates one or more derived flow-related metrics computed from the electrical bioimpedance measurements over time. In the illustrated embodiment, the user interface 400 includes a graphical representation of estimated blood flow rate values derived from the BioZ signal, for example using impedance plethysmography techniques executed by the controller 200. The derived flow metrics may be expressed in volumetric units, such as liters per minute, and presented as discrete or continuous values over a selected time window. In some embodiments, the flow rate estimates are generated during periods of reduced motion, as determined by the accelerometer 350, to improve signal reliability. The flow-related visualization may be used to assess relative changes in peripheral blood flow, pulse amplitude variability, or other hemodynamic trends over time.

In some embodiments, the graphical representations shown in FIG. 8 are displayed concurrently on the user interface 400 to provide a multi-modal view of cardiovascular and hemodynamic activity. The ECG waveform, bioimpedance waveform, and derived flow metrics may be time-aligned to enable visual correlation between cardiac electrical activity, arterial pulse dynamics, and estimated blood flow parameters. Such visualizations may support real-time monitoring, retrospective review, trend analysis, or clinical decision support.

In further embodiments, the displayed signals and metrics may be stored locally or transmitted to a remote system for further analysis, reporting, or integration with electronic health records.

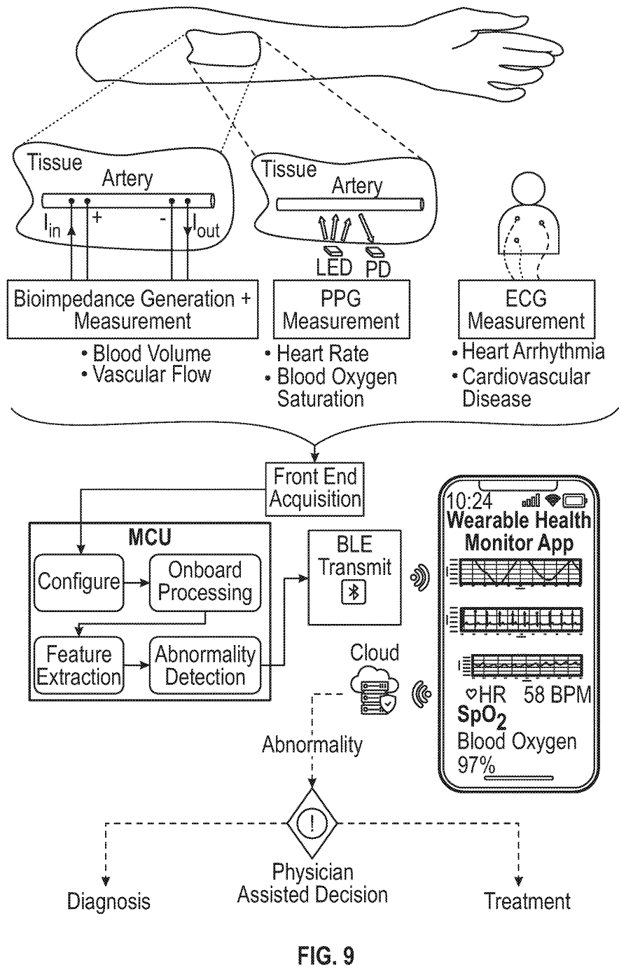

FIG. 9 illustrates the overall system for measuring and processing physiological signals, specifically ECG, PPG, and EBI, in accordance with the present disclosure. In particular, FIG. 9 illustrates an integrated end-to-end wearable health monitoring system, in accordance with the present disclosure, that combines multiple physiological sensing modalities with onboard processing, wireless communication, and clinical decision support.

FIG. 9 shows a wearable device positioned on a user's arm, where different sensing subsystems operate concurrently. A bioimpedance (EBI) subsystem measures blood volume and vascular flow through current injection and voltage sensing across tissue, while an optical photoplethysmography (PPG) subsystem uses an LED and photodiode (PD) to measure heart rate and blood oxygen saturation. In parallel, an electrocardiogram (ECG) subsystem captures electrical cardiac activity to detect conditions such as arrhythmia and cardiovascular disease. These sensing modalities collectively provide complementary physiological insights from the same anatomical region.

The acquired signals are processed through a front-end acquisition module and then provided to a microcontroller unit (MCU), which performs configuration, onboard processing, feature extraction, and abnormality detection. The processed data is transmitted via a Bluetooth Low Energy (BLE) interface to an external device, such as a mobile application (e.g., a wearable health monitor app), where real-time metrics such as heart rate (e.g., 58 BPM) and blood oxygen saturation (e.g., 97%) are displayed. The system may also transmit data to a cloud platform for further analysis.

FIG. 9 further depicts a broader healthcare workflow in which detected abnormalities can be escalated to cloud-based analysis and physician-assisted decision-making, ultimately supporting diagnosis and treatment. This architecture highlights a closed-loop system in which multimodal physiological data acquisition, local and remote processing, and clinical intervention are integrated to enable continuous, real-time monitoring and improved patient outcomes, in accordance with the present disclosure.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

The embodiments disclosed herein are examples of the disclosure and may be embodied in various forms. For instance, although certain embodiments herein are described as separate embodiments, each of the embodiments herein may be combined with one or more of the other embodiments herein. Specific structural and functional details disclosed herein are not to be interpreted as limiting, but as a basis for the claims and as a representative basis for teaching one skilled in the art to variously employ the present disclosure in virtually any appropriately detailed structure. Like reference numerals may refer to similar or identical elements throughout the description of the figures.

The phrases “in an embodiment,” “in embodiments,” “in various embodiments,” “in some embodiments,” or “in other embodiments” may each refer to one or more of the same or different example embodiments provided in the present disclosure. A phrase in the form “A or B” means “(A), (B), or (A and B).” A phrase in the form “at least one of A, B, or C” means “(A); (B); (C); (A and B); (A and C); (B and C); or (A, B, and C).”

It should be understood that the foregoing description is only illustrative of the present disclosure. Various alternatives and modifications can be devised by those skilled in the art without departing from the disclosure. Accordingly, the present disclosure is intended to embrace all such alternatives, modifications, and variances. The embodiments described with reference to the attached drawing figures are presented only to demonstrate certain examples of the disclosure. Other elements, steps, methods, and techniques that are insubstantially different from those described above and/or in the appended claims are also intended to be within the scope of the disclosure.

What is claimed is:

1. A system for monitoring hemodynamic signals of a user, the system comprising:
a wearable device configured to be worn on a limb of the user, the wearable device comprising:
an electrode array comprising one or more current generation electrodes and one or more voltage detection electrodes;
one or more electrocardiogram (ECG) electrodes configured to acquire biopotential signals of the user;
an optical sensing subsystem comprising a light-emitting device configured to emit light toward skin tissue of the user and one or more photodiodes configured to detect light reflected from and/or transmitted through the skin tissue;
an accelerometer configured to detect motion of the user;
a processor; and
a memory, including instructions, stored thereon, when executed by the processor, cause the wearable device to:
generate a photoplethysmography (PPG) signal based on signals detected by the one or more photodiodes;
acquire an ECG signal using the one or more ECG electrodes and an electrical bioimpedance (EBI) signal using the electrode array; and
process the ECG signal, the PPG signal, and the EBI signal based at least in part on motion detected by the accelerometer to compensate for motion-related effects and generate one or more updated physiological signals.

2. The system of claim 1, wherein the instructions, when executed by the processor, further cause the device to temporally synchronize the ECG signal, the PPG signal, and the EBI signal to generate time-aligned physiological data.

3. The system of claim 1, wherein the optical sensing subsystem is configured to perform ambient light cancellation prior to generation of the PPG signal.

4. The system of claim 1, wherein the accelerometer comprises a multi-axis accelerometer, and wherein the controller is configured to compensate for motion-related effects using acceleration data measured along multiple axes.

5. The system of claim 1, wherein the instructions, when executed by the processor, further causes the device to generate an impedance plethysmography (IPG) signal by computing a time derivative of the acquired EBI signal.

6. The system of claim 1, wherein the instructions, when executed by the processor, further causes the device to calculate pulse transit time (PTT) based on a temporal relationship between a feature of the ECG signal and a corresponding feature of at least one of the PPG signal or the EBI signal.

7. The system of claim 6, wherein the instructions, when executed by the processor, further causes the device to estimate systolic blood pressure (SBP) based at least in part on the calculated pulse transit time.

8. The system of claim 1, wherein the electrode array, the light-emitting device of the optical sensing subsystem, and the accelerometer are co-located within a common housing of the wearable device.

9. The system of claim 1, wherein the wearable device is configured to be worn on a forearm of the user, and wherein a bottom surface of the wearable device is configured to be positioned against an inner surface of the forearm during use.

10. The system of claim 1, wherein the wearable device further includes a wireless network interface configured to transmit the one or more updated physiological signals to a mobile computing device executing an application configured to display physiological waveforms or hemodynamic metrics.

11. A method for monitoring hemodynamic signals using a wearable device, comprising:
illuminating skin tissue of the user with one or more wavelengths of light emitted from a light-emitting device of an optical sensing subsystem of the wearable device;
measuring, by one or more photodiodes of the optical sensing subsystem, light reflected from and/or transmitted through the skin tissue;
generating, by a controller of the wearable device, a photoplethysmography (PPG) signal based on the measured light reflected from or transmitted through the skin tissue;
acquiring an electrocardiogram (ECG) signal of the user using one or more ECG electrodes of the wearable device;
acquiring an electrical bioimpedance (EBI) signal of the user using an electrode array of the wearable device;
detecting motion of the user using an accelerometer of the wearable device during acquisition of the ECG signal, the PPG signal, and the EBI signal;
processing, by the controller, the ECG signal, the PPG signal, and the EBI signal based at least in part on the detected motion to reduce motion-related artifacts and generate updated physiological signals; and
transmitting the one or more updated physiological signals to a user interface.

12. The method of claim 11, wherein processing the ECG signal, the PPG signal, and the EBI signal comprises temporally synchronizing the ECG signal, the PPG signal, and the EBI signal to generate time-aligned physiological data.

13. The method of claim 11, further comprising performing ambient light cancellation on optical signals acquired by the optical sensing subsystem prior to generating the PPG signal.

14. The method of claim 11, wherein detecting motion of the user comprises measuring multi-axis acceleration using the accelerometer, and wherein processing comprises compensating for motion-related effects using the measured acceleration.

15. The method of claim 11, further comprising generating an impedance plethysmography (IPG) signal by computing a time derivative of the acquired EBI signal.

16. The method of claim 11, further comprising calculating pulse transit time (PTT) based on a temporal relationship between a feature of the ECG signal and a corresponding feature of at least one of the PPG signal or the EBI signal.

17. The method of claim 16, further comprising estimating systolic blood pressure (SBP) based at least in part on the calculated pulse transit time.

18. The method of claim 11, wherein the electrode array, the light-emitting device of the optical sensing subsystem, and the accelerometer are co-located within a common housing of the wearable device.

19. The method of claim 11, wherein the wearable device is configured to be worn on a forearm of the user, and wherein a bottom surface of the wearable device is positioned against an inner surface of the forearm during use.

20. The method of claim 11, further comprising displaying, on a display of the user interface, one or more of the updated physiological signals or one or more hemodynamic metrics derived therefrom.

21. The method of claim 11, wherein wirelessly transmitting comprises transmitting the updated physiological signals to a mobile computing device executing an application configured to present physiological waveforms or trends.
